(19) United States
(12) Patent Application Publication
(10) Pub. No.: US 2020/0163616 A1
(43) Pub. Date: May 28, 2020

(54) DEVICES, SYSTEMS, AND METHODS TO MONITOR SUBJECT COMPLIANCE WITH A PHARMACEUTICAL TREATMENT REGIMEN

(71) Applicant: Jacqueline Mbwille Sakaya, Belleville, MI (US)

(72) Inventor: Jacqueline Mbwille Sakaya, Belleville, MI (US)

(21) Appl. No.: 16/696,035

(22) Filed: Nov. 26, 2019

Related U.S. Application Data
(60) Provisional application No. 62/772,350, filed on Nov. 28, 2018.

(51) Int. Cl. A61B 5/00; A61B 5/1477; A61B 5/1455; A61B 5/0205; A61B 5/01; G01N 33/543

(52) U.S. Cl. CPC A61B 5/4833; A61B 5/0022; A61B 5/1477; A61B 5/1455; A61B 5/02427; A61B 5/01; A61B 5/747; G01N 33/54366; A61B 5/0205

(57) ABSTRACT

Monitoring devices, systems including the same, and methods of monitoring subject compliance are described herein. A method to monitor compliance with a pharmaceutical treatment regimen includes obtaining, via an autosampler of a wearable medical device, a biological sample, the wearable medical device including the autosampler in fluid communication with the analyte detector. The method further includes determining a concentration of an analyte in the biological sample, which correlates with a probability that the subject received a bolus of the pharmaceutical within a period of time prior to obtaining the biological sample. The method further includes determining whether the concentration of the analyte suggests that the subject received the bolus of the pharmaceutical within the period of time and electronically transmitting a message indicating that the concentration suggests that the subject either received or did not receive the bolus of the pharmaceutical within the period of time.

### CROSS-REFERENCE TO RELATED APPLICATION

The present application claims the priority benefit of U.S. Provisional Patent Application Ser. No. 62/772,350, filed Nov. 28, 2018 and entitled “Methods to Monitor Patient Compliance with a Pharmaceutical Treatment Regimen,” the entire contents of which is incorporated herein in its entirety.

### BACKGROUND

Noncompliance with pharmaceutical treatment regimens is a major cause of preventable illness and/or mortality. Subjects with pre-existing mental impairment and/or subjects receiving pharmaceuticals that cause mental impairment may present an elevated risk of non-compliance, particularly when pharmaceuticals are intended for self-administration. Many such subjects receive multiple different pharmaceuticals having disparate administration regimens, which confounds compliance. Subjects risk mistakenly omitting doses, self-administering multiples of a given dose, or self-administering the wrong pharmaceutical in error. The antiquated use of pill counting remains the gold standard to monitor subject compliance, but pill counts are no panacea. Improved methods of monitoring subject compliance with self-administered pharmaceutical treatment regimens are therefore desirable.

### SUMMARY

In one aspect A1, a method to monitor compliance with a pharmaceutical treatment regimen includes obtaining, via an autosampler of a wearable medical device, a biological sample from a subject. The wearable medical device includes the autosampler in fluid communication with the analyte detector. The subject is receiving a pharmaceutical that is formulated for self-administration. The method further includes determining, via the analyte detector of the wearable medical device, a concentration of an analyte in the biological sample. The concentration of the analyte correlates with a probability that the subject received a bolus of the pharmaceutical within a period of time prior to obtaining the biological sample. The method further includes determining whether the concentration of the analyte suggests that the subject received the bolus of the pharmaceutical within the period of time. The method further includes electronically transmitting a message indicating that the concentration of the analyte suggests that the subject either received the bolus of the pharmaceutical within the period of time or did not receive the bolus of the pharmaceutical within the period of time.

Another aspect A2 includes the method of aspect A1, further including determining whether the concentration of the analyte suggests that the concentration of the pharmaceutical in the subject is higher than a predetermined concentration and electronically transmitting a message that indicates that the concentration of the analyte suggests that the concentration of the pharmaceutical in the subject is higher than the predetermined concentration.

Another aspect A3 includes the method of either of aspects A1-A2, wherein the pharmaceutical has a psychoactive effect.

Another aspect A4 includes the method of any one of aspects A1-A3, wherein the pharmaceutical is capable of crossing the blood-brain barrier, and the pharmaceutical is a central nervous system stimulant or a central nervous system depressant.

Another aspect A5 includes the method of any one of aspects A1-A4, wherein the pharmaceutical has one or more side effects, the pharmaceutical has an administration regimen, and the one or more side effects reduce a probability that the subject will comply with the administration regimen.

Another aspect A6 includes the method of any one of aspects A1-A5, wherein the pharmaceutical has an administration regimen, the subject has a mental condition, and the mental condition reduces a probability that the subject will comply with the administration regimen.

Another aspect A7 includes the method of any one of aspects A1-A6, the method further including contacting the analyte with a detection molecule in the analyte detector, the detection molecule specifically binding the analyte.

Another aspect A8 includes the method of aspect A7, wherein the detection molecule and the analyte have a dissociation constant, and the dissociation constant is less than 10 μM.

Another aspect A9 includes the method of aspect A7 or aspect A8, wherein the detection molecule includes a polypeptide or a nucleic acid.

Another aspect A10 includes the method of aspect A9, wherein the detection molecule consists of or includes an antibody, an antigen-binding portion of an antibody, or an aptamer.

Another aspect A11 includes the method of any one of aspects A7-A10, further including measuring a change in magnitude of a current, a resistance, or a voltage of a circuit in the analyte detector, wherein the change in magnitude correlates with the concentration of the analyte in the biological sample.

Another aspect A12 includes the method of any one of aspects A1-A11, further including detecting an amount of light via a light detector in the analyte detector for a second period of time after obtaining the biological sample, wherein the amount of light either correlates or inversely correlates with the concentration of the reporting molecule.

Another aspect A13 includes the method of any one of aspects A1-A12, wherein the biological sample includes blood, blood plasma, blood serum, sweat, or interstitial fluid.

Another aspect A14 includes the method of any one of aspects A1-A13, further including drawing the biological sample through a central lumen of a needle of the autosampler.

Another aspect A15 includes the method of aspect A14, further including inserting the needle into a skin of the subject.

Another aspect A16 includes the method of any one of aspects A1-A15, wherein determining whether the concentration of the analyte that the subject received the bolus of the pharmaceutical within the period of time includes determining, via a processing device of the wearable medical device, whether the concentration of the analyte that the subject received the bolus of the pharmaceutical within the period of time.

Another aspect A17 includes the method of any one of aspects A1-A15, wherein determining whether the concentration of the analyte that the subject received the bolus of the pharmaceutical within the period of time includes determining, via a computing device remotely located from the wearable medical device and communicatively coupled to the wearable medical device, whether the concentration of the analyte that the subject received the bolus of the pharmaceutical within the period of time.

In another aspect A18, a method to monitor compliance with a pharmaceutical treatment regimen includes identifying a subject who presents a risk of non-compliance with an administration regimen for a self-administered pharmaceutical, providing a wearable medical device comprising an autosampler and an analyte detector to the subject, the autosampler in fluid communication with the analyte detector, and obtaining a biological sample from the subject. The autosampler obtains the biological sample from the subject. The subject is receiving the pharmaceutical. The method further includes determining, via the analyte detector of the wearable medical device, a concentration of an analyte in the biological sample. The concentration of the analyte correlates with a probability that the subject received a bolus of the pharmaceutical within a period of time prior to obtaining the biological sample. The method further includes determining whether the concentration of the analyte suggests that the subject received the bolus of the pharmaceutical within the period of time. The method further includes electronically transmitting a message indicating that the concentration of the analyte suggests that the subject either received the bolus of the pharmaceutical within the period of time or did not receive the bolus of the pharmaceutical within the period of time.

In yet another aspect A19, a wearable medical device to monitor compliance with a pharmaceutical treatment regimen includes an autosampler, an analyte detector fluidly coupled to the autosampler, a processing device communicatively coupled to the analyte detector and the autosampler, and a non-transitory storage medium communicatively coupled to the processing device. The non-transitory storage medium includes one or more programming instructions thereon that, when executed, cause the processing device to direct the autosampler to obtain a biological sample from a subject that is receiving a pharmaceutical that is formulated for self-administration. The non-transitory storage medium further includes one or more programming instructions thereon that, when executed, cause the processing device to direct the analyte detector to determine a concentration of an analyte in the biological sample. The concentration of the analyte correlates with a probability that the subject received a bolus of the pharmaceutical within a period of time prior to obtaining the biological sample. The non-transitory storage medium further includes one or more programming instructions thereon that, when executed, cause the processing device to determine whether the concentration of the analyte suggests that the subject received the bolus of the pharmaceutical within the period of time. The non-transitory storage medium further includes one or more programming instructions thereon that, when executed, cause the processing device to transmit a message indicating that the concentration of the analyte suggests that the subject either received the bolus of the pharmaceutical within the period of time or did not receive the bolus of the pharmaceutical within the period of time.

Another aspect A20 includes the wearable medical device of aspect A19, wherein the one or more programming instructions, when executed, further cause the processing device to determine whether the concentration of the analyte suggests that the concentration of the pharmaceutical in the subject is higher than a predetermined concentration and electronically transmit a message that indicates that the concentration of the analyte suggests that the concentration of the pharmaceutical in the subject is higher than the predetermined concentration.

Another aspect A21 includes the wearable medical device of any one of aspects A19-A20, wherein the analyte detector includes a detection molecule that specifically binds to the analyte when the analyte is contacted with the detection molecule.

Another aspect A22 includes the wearable medical device of aspect A21, wherein the detection molecule includes a polypeptide or a nucleic acid.

Another aspect A23 includes the wearable medical device of aspect A22 wherein the detection molecule includes an antibody, an antigen-binding portion of an antibody, or an aptamer.

Another aspect A24 includes the wearable medical device of any one of aspects A21-A23, wherein the analyte detector includes a circuit, the analyte detector includes an ammeter, ohmmeter, or voltmeter, the circuit is in electrical communication with the ammeter, ohmmeter, or voltmeter, the circuit includes a current, a resistance, and a voltage, each of the current, the resistance, and the voltage includes a magnitude, contacting the analyte with the detection molecule changes the magnitude of the current, the resistance, or the voltage to produce a change in magnitude, and the one or more programming instructions, when executed, further cause the processing device to direct the analyte detector to measure the change in magnitude, the change in magnitude correlating with the concentration of the analyte in the biological sample.

Another aspect A25 includes the wearable medical device of any one of aspects A19-A24, wherein the analyte detector includes a light source, a reporting molecule, a detection chamber, and a light detector, the analyte detector is configured such that the concentration of the analyte in the biological sample correlates with a concentration of the reporting molecule in the detection chamber, the reporting molecule absorbs, transmits, scatters, fluoresces, or phosphoresces light, and the one or more programming instructions, when executed, further cause the processing device to direct the analyte detector to cause the light source to irradiate the detection chamber with light from the light source, direct the analyte detector to cause the light detector to detect light from the detection chamber the period of time after obtaining the biological sample, and determine that the amount of light detected by the light detector either correlates or inversely correlates with the concentration of the reporting molecule in the detection chamber.

Another aspect A26 includes the wearable medical device of any one of aspects A19-A25, wherein the autosampler includes a needle, the needle includes a central lumen, the central lumen of the needle is in fluid communication with the analyte detector, and the one or more programming instructions, when executed, further cause the processing device to direct the autosampler to draw the biological sample through the central lumen of the needle.

Another aspect A27 includes the wearable medical device of aspect A26, wherein the programming instructions, when executed, further cause the processing device to transmit a command to insert the needle into the skin of the subject.

In yet another aspect A28, a wearable medical device to monitor compliance with a pharmaceutical treatment regimen includes an autosampler, an analyte detector fluidly coupled to the autosampler, and means for directing the autosampler to obtain a biological sample from a subject that is receiving a pharmaceutical that is formulated for self-administration, directing the analyte detector to determine a concentration of an analyte in the biological sample, wherein the concentration of the analyte correlates with a probability that the subject received a bolus of the pharmaceutical within a period of time prior to obtaining the biological sample, determining whether the concentration of the analyte suggests that the subject received the bolus of the pharmaceutical within the period of time, and transmitting a message indicating that the concentration of the analyte suggests that the subject either received the bolus of the pharmaceutical within the period of time or did not receive the bolus of the pharmaceutical within the period of time.

In yet another aspect A29, a monitoring system to monitor compliance with a pharmaceutical treatment regimen, the monitoring system including a wearable medical device having an autosampler and an analyte detector fluidly coupled to the autosampler. The autosampler is configured to obtain a biological sample from a subject that is receiving a pharmaceutical that is formulated for self-administration and the analyte detector is configured to determine a concentration of an analyte in the biological sample. The concentration of the analyte correlates with a probability that the subject received a bolus of the pharmaceutical within a period of time prior to obtaining the biological sample. The monitoring system further includes a computing device that is remote from the wearable medical device and communicatively coupled to the medical device. The computing device is configured to receive data from the analyte detector corresponding to the determined concentration of the analyte in the biological sample, determine whether the concentration of the analyte suggests that the subject received the bolus of the pharmaceutical within the period of time, and transmit a message indicating that the concentration of the analyte suggests that the subject either received the bolus of the pharmaceutical within the period of time or did not receive the bolus of the pharmaceutical within the period of time.

Another aspect A30 includes the monitoring system of aspect A29, wherein the analyte detector includes a detection molecule that specifically binds to the analyte when the analyte is contacted with the detection molecule.

Another aspect A31 includes the monitoring system of aspect A30, wherein the detection molecule includes a polypeptide or a nucleic acid.

Another aspect A32 includes the monitoring system of aspect A31, wherein the detection molecule includes an antibody, an antigen-binding portion of an antibody, or an aptamer.

Another aspect A33 includes the monitoring system of any one of aspects A29-A32, wherein the analyte detector includes a circuit, the analyte detector includes an ammeter, ohmmeter, or voltmeter, the circuit is in electrical communication with the ammeter, ohmmeter, or voltmeter, the circuit includes a current, a resistance, and a voltage, each of the current, the resistance, and the voltage includes a magnitude, contacting the analyte with the detection molecule changes the magnitude of the current, the resistance, or the voltage to produce a change in magnitude, and the analyte detector is further configured to measure the change in magnitude, the change in magnitude correlating with the concentration of the analyte in the biological sample.

Another aspect A34 includes the monitoring system of any one of aspects A29-A33, wherein the analyte detector includes a light source, a reporting molecule, a detection chamber, and a light detector; the concentration of the analyte in the biological sample correlates with a concentration of the reporting molecule in the detection chamber, the reporting molecule absorbs, transmits, scatters, fluoresces, or phosphoresces light, the analyte detector is further configured to cause the light source to irradiate the detection chamber with light from the light source, cause the light detector to detect light from the detection chamber the period of time after obtaining the biological sample and determine that the amount of light detected by the light detector either correlates or inversely correlates with the concentration of the reporting molecule in the detection chamber.

Another aspect A35 includes the monitoring system of any one of aspects A29-A34 wherein the autosampler includes a needle, the needle includes a central lumen, the central lumen of the needle is in fluid communication with the analyte detector, and the autosampler is further configured to draw the biological sample through the central lumen of the needle.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 depicts an illustrative monitoring system to monitor compliance with a pharmaceutical treatment regimen according to one or more embodiments shown and described herein;

FIG. 2 depicts illustrative internal components of a wearable medical device to monitor compliance with a pharmaceutical treatment regimen according to one or more embodiments shown and described herein; and

FIG. 3 depicts illustrative internal components of a computing device according to one or more embodiments shown and described herein.

### DETAILED DESCRIPTION

Various aspects of the present disclosure relate to devices, systems, and methods to monitor subject compliance with a pharmaceutical treatment regimen. Specifically, the aspects of the present disclosure generally relate to subjects who present a risk of non-compliance with an administration regimen for a self-administered pharmaceutical, which include, but are not limited to, subjects who display a risk of cognitive impairment such as subjects presenting with mild cognitive impairment and Alzheimer's disease. Other diseases and conditions that cause cognitive impairment include amyotrophic lateral sclerosis, Parkinson's disease, Lewy-Body disease, Huntington's disease, multiple sclerosis, frontotemporal degeneration, acquired immune deficiency disorder, meningitis, benign and malignant brain tumors, ischemic and hemorrhagic stroke, cerebral small vessel disease, head injury, traumatic brain injury, penetrating head injuries, amnesia, dementia, delirium, psychiatric illness, psychosis, psychoactive drug use, withdrawal from chronic exposure to a psychoactive drug, exposure to toxic substances such as heavy metals or pesticides, Wernicke-Korsakoff syndrome, vitamin deficiency, malnutrition, metabolic imbalance, hormonal imbalance, and aging.

A subject may present with mild cognitive impairment, Alzheimer's disease, amyotrophic lateral sclerosis, Parkinson's disease, Lewy-Body disease, Huntington's disease, multiple sclerosis, frontotemporal degeneration, acquired immune deficiency disorder, meningitis, a benign or malignant brain tumor, ischemic or hemorrhagic stroke, cerebral small vessel disease, head injury, traumatic brain injury, a penetrating head injury, amnesia, dementia, delirium, psychiatric illness, psychosis, psychoactive drug use, withdrawal from chronic exposure to a psychoactive drug, exposure to a toxic substance such as a heavy metals or pesticides, Wernicke-Korsakoff syndrome, vitamin deficiency, malnutrition, metabolic imbalance, hormonal imbalance, and aging. A subject according to the instant disclosure is human.

In the embodiments described herein, it may be necessary to identify a subject who presents with cognitive impairment, such as for example, identifying a subject who presents with mental illness, identifying a subject who presents with mild cognitive impairment, Alzheimer's disease, amyotrophic lateral sclerosis, Parkinson's disease, Lewy-Body disease, Huntington's disease, multiple sclerosis, frontotemporal degeneration, acquired immune deficiency disorder, meningitis, a benign or malignant brain tumor, ischemic or hemorrhagic stroke, cerebral small vessel disease, head injury, traumatic brain injury, a penetrating head injury, amnesia, dementia, delirium, psychiatric illness, psychosis, psychoactive drug use, withdrawal from chronic exposure to a psychoactive drug, exposure to a toxic substance such as a heavy metals or pesticides, Wernicke-Korsakoff syndrome, vitamin deficiency, malnutrition, metabolic imbalance, hormonal imbalance, and aging.

Various pharmaceutical treatment regimens present compliance risks. Pharmaceuticals that are associated with a risk of cognitive impairment, for example, are often associated with a risk of non-compliance. Many such pharmaceuticals display a psychoactive effect. A pharmaceutical may nevertheless be associated with a risk of cognitive impairment even if the pharmaceutical does not display a psychoactive effect, for example, if the pharmaceutical alters homeostasis such as the homeostasis of one or more fluids, ions, glucose, or hormones of a subject. Insulin, for example, may have an indirect psychoactive effect by reducing blood glucose levels because the brain relies upon glucose as its preferential energy source.

Accordingly, it may be necessary to identify that a subject is receiving a pharmaceutical that is associated with a risk of cognitive impairment, such as a pharmaceutical that has a psychoactive effect.

A subject of the instant disclosure is typically receiving a pharmaceutical that is formulated for self-administration. A subject may be receiving a pharmaceutical that is formulated, for example, for oral administration, sublingual administration, buccal administration, inhalational administration, insufflation, transdermal administration, transmucosal administration, sub-cutaneous injection, or intramuscular injection.

In some embodiments, a subject is receiving one or more pharmaceutical(s) selected from abiraterone acetate, acamprosate calcium, acebutolol hydrochloride, acetaminophen, acetazolamide, acetohexamide, acetophenazine maleate, acetylcysteine, acetyldigitoxin, albuterol sulfate, alendronate sodium, aliskiren hemifumarate, alkavervir, allopurinol, alprazolam, alseroxylon, amantadine hydrochloride, ambenonium chloride, amiloride hydrochloride, aminoglutethimide, aminophylline, amiodarone hydrochloride, amitriptyline hydrochloride, amlodipine besylate, amlodipine maleate, amodiaquine hydrochloride, amoxapine, amoxicillin, amphetamine aspartate, amphetamine resin complex, amphetamine sulfate, amphotericin b, ampicillin, ampicillin trihydrate, amprenavir, anagrelide hydrochloride, anileridine hydrochloride, anisindione, anisotropine methylbromide, aripiprazole, armodafinil, aspirin, atazanavir sulfate, atomoxetine hydrochloride, atovaquone, atropine sulfate, azatadine maleate, azathioprine, azithromycin, bacampicillin hydrochloride, baclofen, benazepril hydrochloride, bendroflumethiazide, bentiromide, benzonatate, benzphetamine hydrochloride, benzthiazide, benztropine mesylate, bepridil hydrochloride, betamethasone, betaxolol hydrochloride, bethanechol chloride, bethanidine sulfate, biperiden hydrochloride, bisacodyl, bisoprolol fumarate, bromodiphenhydramine hydrochloride, brompheniramine maleate, buclizine hydrochloride, bupropion hydrochloride, buspirone hydrochloride, butabarbital sodium, butalbital, cabergoline, captopril, carbenicillin indanyl sodium, carbinoxamine maleate, carisoprodol, carphenazine maleate, carprofen, carteolol hydrochloride, cefaclor, cefadroxil/cefadroxil hemihydrate, cefdinir, cefditoren pivoxil, cefixime, cefpodoxime proxetil, cefprozil, ceftibuten dihydrate, cefuroxime axetil, cephalexin, cephalexin hydrochloride, cephaloglycin, cephradine, cerivastatin sodium, cetirizine hydrochloride, chenodiol, chlophedianol hydrochloride, chlordiazepoxide, chlormezanone, chloroquine phosphate, chlorothiazide, chlorotriani sene, chlorphenesin carbamate, chlorpheniramine maleate, chlorpheniramine polistirex, chlorphentermine hydrochloride, chlorpromazine hydrochloride, chlorpropamide, chlorprothixene, chlorthalidone, chlorzoxazone, cholestyramine, cimetidine, cimetidine hydrochloride, cinacalcet hydrochloride, cinoxacin, cisapride monohydrate, citalopram hydrobromide, clarithromycin, clavulanate potassium, clemastine fumarate, clidinium bromide, clindamycin hydrochloride, clobazam, clofazimine, clofibrate, clomiphene citrate, clomipramine hydrochloride, clonazepam, clonidine, clonidine hydrochloride, clorazepate dipotassium, clotrimazole, cloxacillin sodium, clozapine, codeine phosphate, codeine polistirex, colchicine, colesevelam hydrochloride, colistin sulfate, cortisone acetate, cromolyn sodium, cryptenamine tannates, cyclacillin, cyclobenzaprine hydrochloride, cyclophosphamide, cyclosporine, cyclothiazide, cycrimine hydrochloride, cyproheptadine hydrochloride, dalfampridine, danazol, darunavir ethanolate, demeclocycline hydrochloride, deserpidine, desipramine hydrochloride, desmopressin acetate, desogestrel, dexamethasone, dexbrompheniramine maleate, dextroamphetamine resin complex, dextroamphetamine saccharate, dextroamphetamine sulfate, dextromethorphan hydrobromide, dextrothyroxine sodium, diatrizoate sodium, diazepam, diazoxide, dichlorphenamide, diclofenac potassium, diclofenac sodium, dicloxacillin sodium, dicumarol, dicyclomine hydrochloride, didanosine, diethylcarbamazine citrate, diethylpropion hydrochloride, diethylstilbestrol, diethylstilbestrol diphosphate, difenoxin hydrochloride, diflunisal, dihydrocodeine bitartrate, diltiazem hydrochloride, diltiazem malate, diphemanil methylsulfate, diphenhydramine hydrochloride, diphenidol hydrochloride, diphenoxylate hydrochloride, diphenylpyraline hydrochloride, dipyridamole, dirithromycin, disulfiram, divalproex sodium, dolasetron mesylate, donepezil hydrochloride, doxepin hydrochloride, doxycycline hyclate, doxylamine succinate, duloxetine hydrochloride, dydrogesterone, dyphylline, edetate calcium disodium, efavirenz, enalapril maleate, enoxacin, eplerenone, ergoloid mesylates, ergotamine tartrate, erythromycin, erythromycin estolate, erythromycin ethylsuccinate, estazolam, estropipate, ethacrynic acid, ethchlorvynol, ethinamate, ethinyl estradiol, ethopropazine hydrochloride, ethotoin, ethoxzolamide, ethylestrenol, ethynodiol diacetate, etidronate disodium, etodolac, etoposide, etretinate, ezetimibe, ezogabine, famciclovir, famotidine, felodipine, fenofibrate, fenoprofen calcium, fentanyl citrate, ferumoxsil, fexofenadine hydrochloride, flavoxate hydrochloride, flecainide acetate, fludarabine phosphate, fludrocortisone acetate, fluoxetine hydrochloride, fluoxymesterone, fluphenazine hydrochloride, fluprednisolone, flurazepam hydrochloride, flurbiprofen, flutamide, fluvastatin sodium, fluvoxamine maleate, fosinopril sodium, frovatriptan succinate, furazolidone, furosemide, gabapentin, galantamine hydrobromide, ganciclovir, gefitinib, gemfibrozil, glipizide, glutethimide, glyburide, glycopyrrolate, granisetron hydrochloride, grepafloxacin hydrochloride, griseofulvin, guaifenesin, guanabenz acetate, guanadrel sulfate, guanethidine monosulfate, guanfacine hydrochloride, halazepam, halofantrine hydrochloride, haloperidol, haloperidol lactate, hetacillin, hexocyclium methylsulfate, homatropine methylbromide, hydralazine hydrochloride, hydrochlorothiazide, hydrocodone bitartrate, hydrocodone polistirex, hydroflumethiazide, hydromorphone hydrochloride, hydroxychloroquine sulfate, hydroxyzine hydrochloride, hydroxyzine pamoate, ibandronate sodium, ibuprofen, imatinib mesylate, imipramine hydrochloride, imipramine pamoate, indapamide, indecainide hydrochloride, indinavir sulfate, indomethacin, iocetamic acid, iohexol, iopanoic acid, ipodate calcium, ipodate sodium, irbesartan, isopropamide iodide, isosorbide, isosorbide dinitrate, isosorbide mononitrate, isotretinoin, isradipine, ivermectin, ketoconazole, ketoprofen, ketorolac tromethamine, labetalol hydrochloride, lamotrigine, lansoprazole, lanthanum carbonate, leucovorin calcium, levamisole hydrochloride, levocarnitine, levofloxacin, levomethadyl acetate hydrochloride, levonorgestrel, levopropoxyphene napsylate anhydrous, levorphanol tartrate, levothyroxine sodium, lidocaine, lidocaine hydrochloride, lincomycin hydrochloride, linezolid, liothyronine sodium, liotrix, lisinopril, lomefloxacin hydrochloride, loperamide hydrochloride, lopinavir, loracarbef, loratadine, losartan potassium, lovastatin, loxapine hydrochloride, loxapine succinate, maprotiline hydrochloride, mazindol, mebendazole, mebutamate, mecamylamine hydrochloride, meclizine hydrochloride, meclofenamate sodium, medroxyprogesterone acetate, mefloquine hydrochloride, megestrol acetate, meloxicam, memantine hydrochloride, menadiol sodium diphosphate, menadione, mepenzolate bromide, meperidine hydrochloride, mephenytoin, mepredni sone, meprobamate, mesalamine, mesoridazine besylate, mestranol, metaproterenol sulfate, metaxalone, metformin hydrochloride, methadone hydrochloride, methamphetamine hydrochloride, methantheline bromide, metharbital, methazolamide, methdilazine, methdilazine hydrochloride, methimazole, methixene hydrochloride, methocarbamol, methotrexate sodium, methoxsalen, methscopolamine bromide, methsuximide, methyclothiazide, methyldopa, methyl ergonovine maleate, methylphenidate hydrochloride, methylprednisolone, methyltestosterone, methyprylon, methysergide maleate, metoclopramide hydrochloride, metolazone, metoprolol fumarate, metoprolol succinate, metoprolol tartrate, metronidazole, metyrapone, mexiletine hydrochloride, midazolam hydrochloride, midodrine hydrochloride, minocycline hydrochloride, minoxidil, mirtazapine, moexipril hydrochloride, molindone hydrochloride, moricizine hydrochloride, morphine sulfate, nabumetone, nadolol, nafcillin sodium, nalidixic acid, naloxone hydrochloride, naltrexone hydrochloride, naproxen, naproxen sodium, nefazodone hydrochloride, nelfinavir mesylate, neomycin sulfate, nicardipine hydrochloride, niclosamide, nifedipine, nilutamide, nimodipine, nisoldipine, nitrofurantoin, nizatidine, norethindrone, norethindrone acetate, norethynodrel, norfloxacin, norgestimate, norgestrel, nortriptyline hydrochloride, nystatin, ofloxacin, omeprazole, orphenadrine citrate, orphenadrine hydrochloride, oseltamivir phosphate, oxacillin sodium, oxamniquine, oxandrolone, oxaprozin potassium, oxazepam, oxprenolol hydrochloride, oxtriphylline, oxybutynin chloride, oxycodone hydrochloride, oxycodone terephthalate, oxymorphone hydrochloride, oxyphenbutazone, oxyphencyclimine hydrochloride, oxyphenonium bromide, oxytetracycline, oxytetracycline hydrochloride, paliperidone, palonosetron hydrochloride, paramethadione, paramethasone acetate, pargyline hydrochloride, paricalcitol, paroxetine hydrochloride, pazopanib hydrochloride, pemoline, penbutolol sulfate, penicillamine, penicillin g benzathine, pentazocine hydrochloride, pentoxifylline, perflubron, pergolide mesylate, perindopril erbumine, perphenazine, phenacemide, phenazopyridine hydrochloride, phendimetrazine tartrate, phenindione, phenmetrazine hydrochloride, phenprocoumon, phensuximide, phentermine hydrochloride, phentermine resin complex, phenylbutazone, phenylephrine hydrochloride, phenylpropanolamine hydrochloride, phenytoin, phenytoin sodium, pinacidil, pindolol, piperacetazine, piperazine citrate, pipobroman, polythiazide, pralidoxime chloride, pramipexole dihydrochloride, pravastatin sodium, prazepam, prazosin hydrochloride, prednisolone, prednisolone acetate, prednisone, primaquine phosphate, primidone, probenecid, probucol, procainamide hydrochloride, prochlorperazine edisylate, prochlorperazine maleate, procyclidine hydrochloride, promazine hydrochloride, promethazine hydrochloride, propafenone hydrochloride, propantheline bromide, propoxyphene hydrochloride, propoxyphene napsylate, propranolol hydrochloride, propylthiouracil, protriptyline hydrochloride, pseudoephedrine hydrochloride, pseudoephedrine polistirex, pseudoephedrine sulfate, pyridostigmine bromide, pyridoxine hydrochloride, pyrimethamine, pyrvinium pamoate, quazepam, quetiapine fumarate, quinestrol, quinethazone, quinidine gluconate, quinidine polygalacturonate, quinidine sulfate, rabeprazole sodium, ramipril, ranitidine bismuth citrate, ranitidine hydrochloride, repaglinide, rescinnamine, reserpine, ribavirin, riluzole, rimantadine hydrochloride, risedronate sodium, risperidone, ritodrine hydrochloride, ritonavir, rivastigmine tartrate, rizatriptan benzoate, rofecoxib, ropinirole hydrochloride, rosiglitazone maleate, rosuvastatin calcium, rufinamide, saquinavir, selegiline hydrochloride, sertraline hydrochloride, sevelamer carbonate, sevelamer hydrochloride, sibutramine hydrochloride, sildenafil citrate, simvastatin, sirolimus, sodium polystyrene sulfonate, sotalol hydrochloride, sparfloxacin, spirapril hydrochloride, stanozolol, stavudine, sulfacytine, sulfadiazine, sulfadoxine, sulfamerazine, sulfameter, sulfamethazine, sulfamethizole, sulfamethoxazole, sulfaphenazole, sulfapyridine, sulfasalazine, sulfinpyrazone, sulfisoxazole, sulfisoxazole acetyl, sulfoxone sodium, sulindac, tacrine hydrochloride, talbutal, tamoxifen citrate, tegaserod maleate, telbivudine, telithromycin, terazosin hydrochloride, terbinafine hydrochloride, terbutaline sulfate, teriflunomide, testolactone, tetracycline hydrochloride, tetracycline phosphate complex, theophylline, theophylline sodium glycinate, thiabendazole, thiethylperazine maleate, thioridazine, thioridazine hydrochloride, thiothixene, thiothixene hydrochloride, thyroglobulin, tiagabine hydrochloride, ticlopidine hydrochloride, tiludronate di sodium, timolol maleate, tizanidine hydrochloride, tocainide hydrochloride, tolazamide, tolbutamide, tolcapone, tolmetin sodium, tolvaptan, topiramate, tramadol hydrochloride, tranexamic acid, trazodone hydrochloride, tretinoin, triamcinolone, triamcinolone diacetate, triamterene, triazolam, trichlormethiazide, triclofos sodium, tridihexethyl chloride, trifluoperazine hydrochloride, triflupromazine, triflupromazine hydrochloride, trihexyphenidyl hydrochloride, trilostane, trimeprazine tartrate, trimethadione, trimethoprim, trioxsalen, tripelennamine citrate, tripelennamine hydrochloride, triprolidine hydrochloride, troglitazone, troleandomycin, trospium chloride, trovafloxacin mesylate, tyropanoate sodium, uracil mustard, ursodiol, valdecoxib, valproic acid, valsartan, vardenafil hydrochloride, venlafaxine hydrochloride, verapamil hydrochloride, veratrum viride root, warfarin potassium, warfarin sodium, zalcitabine, zidovudine, zileuton, ziprasidone hydrochloride, zolpidem tartrate, and zonisamide. An analyte may be any one of the foregoing, an ion of any one of the foregoing, a protonated or deprotonated form of any one of the foregoing, or a metabolite of any one of the foregoing.

In the embodiments described herein, a monitoring device or a monitoring system may be used to monitor compliance with a pharmaceutical treatment regimen. Illustrative examples of such monitoring devices and/or monitoring systems are depicted in the figures and are described in greater detail hereinbelow.

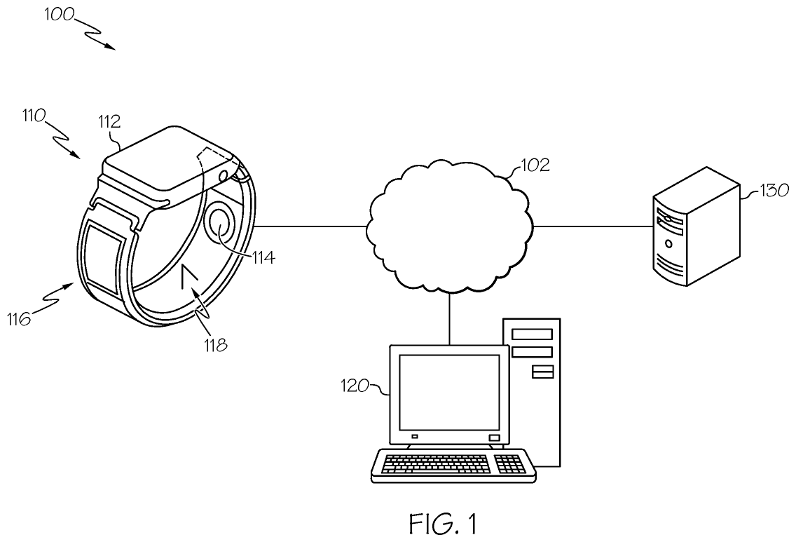

FIG. 1 depicts an illustrative monitoring system 100 that is used to monitor compliance with a pharmaceutical treatment regimen according to various embodiments. The monitoring system 100 generally includes various components communicatively coupled to one another, such as, for example, via a network 102. The various components depicted in the embodiment FIG. 1 include, but are not limited to, a wearable medical device 110, a computing device 120, and/or a server 130 coupled together via the network 102. However, it should be understood that other additional or alternative components may be included in other embodiments, and some components may be omitted in other embodiments. For example, another embodiment may only include the wearable medical device 110 and the computing device 120 communicatively coupled to each other via the network 102 or via a direct connection. Accordingly, it should be understood that while the embodiment of FIG. 1 depicts the network 102, other embodiments may not include the network 102.

The network 102 may include a wide area network (WAN), such as the Internet, a local area network (LAN), a mobile communications network, a public service telephone network (PSTN), a personal area network (PAN), a metropolitan area network (MAN), a virtual private network (VPN), and/or another network. The network 102 may generally be configured to electronically and communicatively connect one or more of the components of the monitoring system 100, including, but not limited to, the wearable medical device 110, the computing device 120, and the server 130. Other components, such as other computing devices, data repositories, or the like, may also be coupled to the network 102 in some embodiments.

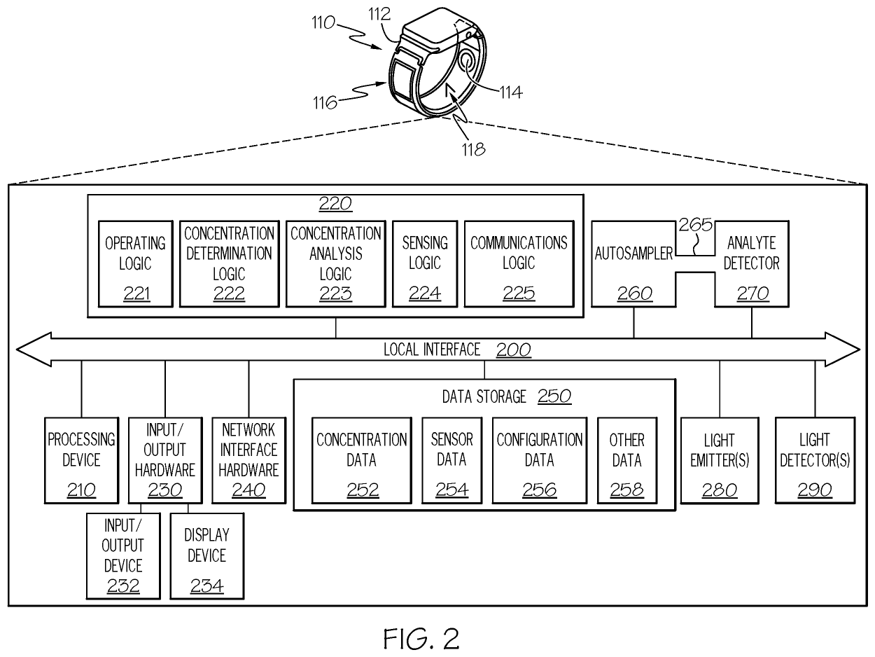

The wearable medical device 110 is generally any device that may be configured to be worn around the wrist, ankle, arm, leg, head, neck, or torso of a subject. The precise positioning of the wearable medical device 110 on a subject is not particularly limiting, and the positioning is instead selected for convenience. Subjects are generally accustomed to wearing devices on their wrists, such as a watch, smart watch, or a wearable health device such as a FITBIT® (Fitbit, Inc., San Francisco Calif.), and devices that are worn around the wrist therefore present certain advantages. The wearable medical device 110 typically includes a fastening mechanism, which may include a buckle, clasp, strap, band, loop, and/or the like that can be made out of any suitable material (e.g., VELCRO® or elastic), although the precise nature of a fastening mechanism is not particularly limiting. In some embodiments, the wearable medical device 110 may be worn on the head of a subject. Additional details regarding the wearable medical device 110 will be described herein with respect to FIG. 2.

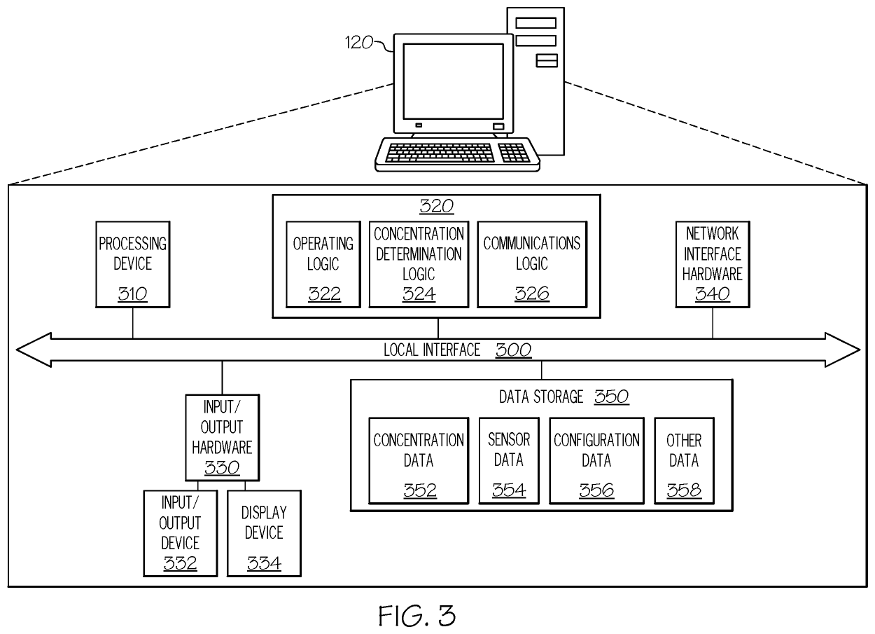

Still referring to FIG. 1, the computing device 120 is generally a device that is remote from the wearable medical device 110, but remains communicatively coupled to the wearable medical device 110 (e.g., via the network 102 and/or via a direct connection) such that data and/or signals can be transmitted therebetween, as described in greater detail herein. In some embodiments, the computing device 120 may be communicatively coupled to the wearable medical device 110 via a wired connection. In other embodiments, the computing device 120 may be communicatively coupled to the wearable medical device 110 via a wireless connection. Additional details regarding the computing device 120 will be described herein with respect to FIG. 3.

Still referring to FIG. 1, the server 130 is generally a device that is communicatively coupled to the wearable medical device 110 and/or the computing device 120 to transmit and/or receive data to/from the wearable medical device 110 and/or the computing device 120. In some embodiments, the server 130 may be a file server that stores data generated as a result of operation of the wearable medical device 110 and/or the computing device 120. In some embodiments, the server 130 may be a database server that maintains a base for the data generated as a result of operation of the wearable medical device 110 and/or the computing device 120, and/or maintains a base for data that may be accessed by the wearable medical device 110 and/or the computing device 120 for the purposes of operating the wearable medical device 110 and/or the computing device 120. The server 130 is generally a computing device that contains components for executing various processes, such as the processes described herein. That is, the server 130 may include at least one or more processing devices and a non-transitory memory component, where the non-transitory memory component includes programming instructions that cause the one or more processing devices to execute the various processes described herein. In some embodiments, the server 130 may include a data storage component that is used for storing data pertaining to the various components of the monitoring system 100 such that the data can be later accessed. In some embodiments, the server 130 may include networking hardware that is used for communicating with the various components of the monitoring system 100. Other components and functionality of the server 130 should generally be understood.

In some embodiments, the monitoring system 100 and/or one or more component thereof may include a location sensor (not shown), or the monitoring system 100 may be configured to receive a signal from a location sensor, wherein the signal includes location information. For example, the location sensor may detect a secondary device of known location or the location sensor may be a global positioning system (GPS). A monitoring system may periodically update its location by obtaining location data from one or more secondary devices. In some embodiments, learning algorithms may be used to create or adjust the rules for determining the location.

Global Navigation Satellite System (GNSS) Time To First Fix (TTFF) may be improved with information from other static or semi-static wireless communication devices including, but not limited to, cell phone towers and Wi-Fi access points. By using a lookup service (e.g., Skyhook), an accurate location can be quickly determined, especially in urban areas comprising abundant cell towers and Wi-Fi routers. In some embodiments, it may be desirable to not have or use cellular, GNSS, and/or Wi-Fi receivers in a monitoring system due to size, power, and/or cost constraints. The monitoring system 100 may nevertheless be able to take advantage of location sensitive secondary computing devices in communication with the monitoring system 100. For example, when the monitoring system 100 is within wireless communication range of a secondary device, such as a smartphone, the monitoring system 100 may download cellular based, Wi-Fi based, and/or GNSS based location data from the secondary device. Indeed, information from any location determining mechanism on one or more secondary devices may be downloaded to the monitoring system 100. Such information may aid in accelerating the TTFF of the monitoring system 100. In such an embodiment, the secondary device may maintain its last known position in memory to communicate to the monitoring system 100 when needed. The smartphone or other secondary device may use Secure User Plane Location (SUPL) to aid in getting a GPS fix. Location data may also be downloaded from the secondary device as described herein.

Referring now to FIG. 2, the wearable medical device 110 may include a local interface 200 (e.g., a bus) that communicatively interconnects the various components, including, but not limited to, a processing device 210, memory 220, input/output hardware 230, network interface hardware 240, a data storage device 250, an autosampler 260, and/or an analyte detector 270.

The local interface 200 is formed from any medium that transmits a signal. As non-limiting examples, the local interface 200 is formed of conductive wires, conductive traces, optical waveguides, or the like. The local interface 200 may also refer to the expanse in which electromagnetic radiation and their corresponding electromagnetic waves are propagated. Moreover, the local interface 200 may be formed from a combination of mediums that transmit signals. In one embodiment, the local interface 200 includes a combination of conductive traces, conductive wires, connectors, and buses that cooperate to permit the transmission of electrical data signals to and from the various components of the wearable medical device 110. Additionally, it is noted that the term “signal” means a waveform (e.g., electrical, optical, magnetic, mechanical or electromagnetic) that travels through a medium, such as DC, AC, sinusoidal-wave, triangular-wave, square-wave, vibration, and the like.

The processing device 210, such as one or more computer processing units (CPU), may be the central processing unit of the wearable medical device 110, performing calculations and logic operations required to execute a program. The processing device 210, alone or in conjunction with one or more of the other elements disclosed in FIG. 2, is an illustrative processing device, computing device, processor, or combination thereof, as such terms are used in this disclosure. As a non-limiting example, processing device 210 may be one of a shared processor circuit, dedicated processor circuit, or group processor circuit. As described herein, the term “shared processor circuit” refers to a single processor circuit that executes some or all machine-readable instructions from the multiple modules. As described herein, the term “group processor circuit” refers to a processor circuit that, in combination with additional processor circuits, executes some or all machine-executable instructions from the multiple modules of one or more non-transitory computer-readable mediums. References to multiple processor circuits encompass multiple processor circuits on discrete dies, multiple processor circuits on a single die, multiple cores of a single processor circuit, multiple threads of a single processor circuit, or a combination of the above.

The memory 220, such as read only memory (ROM) and random access memory (RAM), may constitute illustrative memory devices (i.e., non-transitory, processor-readable storage media). As a non-limiting example, the memory 220 may be one of a shared memory circuit, dedicated memory circuit, or group memory circuit. As described herein, the term “shared memory circuit” refers to a single memory circuit that stores some or all machine-readable instructions from multiple modules, which are described below in further detail. As described herein, the term “group memory circuit” refers to a memory circuit that, in combination with additional memories, stores some or all machine-readable instructions from the multiple modules. Non-limiting examples of the memory 220 include random access memory (including SRAM, DRAM, and/or other types of random access memory), read-only memory (ROM), flash memory, registers, compact discs (CD), digital versatile discs (DVD), and/or other types of storage components.

The memory 220 may include one or more programming instructions thereon that, when executed by the processing device 210, cause the processing device 210 to complete various processes, such as the processes described herein. Optionally, the program instructions may be stored on a tangible computer-readable medium such as a digital disk, flash memory, a memory card, a USB drive, an optical disc storage medium (e.g., Blu-ray™, CD, DVD), and/or other non-transitory processor-readable storage media.

In some embodiments, the program instructions contained on the memory 220 may be embodied as a plurality of software modules, where each module provides programming instructions for completing one or more tasks. For example, as shown in FIG. 2, the memory 220 may contain one or more of operating logic 221, concentration determination logic 222, concentration analysis logic 223, sensing logic 224, and communications logic 225. The operating logic 221 may include an operating system and/or other software for managing components of the wearable medical device 110. The concentration determination logic 222 may include programming instructions for determining a concentration of an analyte in a biological sample using various components of the wearable medical device 110, as described in greater detail herein. The concentration analysis logic 223 may include programming instructions for determining a meaning of a determined concentration, such as, for example, determining whether the concentration suggests that a subject received a bolus of a pharmaceutical within a particular time period, as described in greater detail herein. The sensing logic 224 may include programming instructions for operating the autosampler 260 and/or the analyte detector 270, as described in greater detail herein. The communications logic 225 may include programming instructions for facilitating communications between the wearable medical device 110 and the various other components of the monitoring system 100 (FIG. 1). It should be understood that the various logic modules described herein with respect to FIG. 2 are merely illustrative, and that other logic modules, including logic modules that combine the functionality of two or more of the modules described hereinabove, may be used without departing from the scope of the present application.

Still referring to FIG. 2, the data storage device 250, which may generally be a storage medium that is separate from the memory 220, may contain a data repository for storing electronic data and/or the like relating to a configuration of the wearable medical device 110, information pertaining to a determined concentration and results of analysis, and/or the like. The data storage device 250 may be any physical storage medium, including, but not limited to, a hard disk drive (HDD), memory (e.g., flash memory), removable storage, and/or the like. While the data storage device 250 is depicted as a local device, it should be understood that the data storage device 250 may be a remote storage device that is remotely located from the wearable medical device 110, such as, for example, a server computing device or the like (e.g., the server 130 of FIG. 1). In some embodiments, the data storage device 250 may include or may be coupled to one or more database servers that support NoSQL, MySQL, Oracle, SQL Server, NewSQL, or the like.

Still referring to FIG. 2, illustrative data that may be contained within the data storage device 250 may include, for example, concentration data 252, sensor data 254, configuration data 256, and/or other data 258. Concentration data 252 may include, for example, information pertaining to a determination of a particular concentration of an analyte in a biological sample, as well as data pertaining to a meaning of the particular concentration. The sensor data 254 may include, for example, data relating to information that is sensed by the analyte detector 270. The configuration data 256 may include, for example, data relating to configuring the wearable medical device 110.

The input/output hardware 230 may include a basic input/output system (BIOS) that interacts with hardware of the wearable medical device 110 (such as, for example, an input/output device 232 and/or a display device 234), device drivers that interact with particular components of the wearable medical device 110, one or more operating systems, user applications, background services, background applications, etc.

The display device 234 is generally any liquid crystal display (LCD), light emitting diode (LED) display, electronic ink (e-ink) display, or the like that can display information to a user. In some embodiments, the display device 234 may be configured as an interactive display that can receive user inputs (e.g., a touch screen display or the like). In some embodiments, the display device 234 may be a visual display, such as the screen of a cell phone, computer, TV, or head-mounted display. In some embodiments, the display device 234 may be a cathode ray tube (CRT) display, plasma display panel (PDP), digital light processing (DLP) display, liquid crystal on silicon (LCoS) display, liquid-crystal display (LCD), active-matrix liquid-crystal display (AMLCD), laser video display, light-emitting diode (LED) display, surface-conduction electron-emitter display (SED), field emission display (FED), nano-emissive display (NED), electronic paper display (EPD), organic light-emitting diode (OLED) display, active matrix OLED display, quantum dot display (QDLE), interferometric modulator display (IMOD), laser phosphor display (LPD), or a virtual retinal display. In some embodiments, the display device 234 may be a direct view display or a projected display. In some embodiments, the display device 234 may be controlled by the processing device 210. In some embodiments, the display device 234 may include a transmissive projection display (e.g., where a light source is modulated by an optically active material, backlit with white light). In some embodiments, the display device 234 may include an LCD display. In some embodiments, the display device 234 may include a reflective display (e.g., wherein external light is reflected and modulated by an optically active material. In some embodiments, the display device 234 may include a (DLP) digital light processing display or a liquid crystal on silicon (LCOS) display). A display may include an emissive display, such as a PicoP™ display. In some embodiments, the display device 234 may include an organic light emitting diode (OLED) display.

The input/output device 232 may be hardware components that receive inputs from a user and transmit signals corresponding to the inputs, such as a keyboard, a mouse, a joystick, a touch screen, a remote control, a pointing device, a video input device, an audio input device, a haptic feedback device, and/or the like.

In some embodiments, the display device 234 and the input/output device 232 may be combined into a single device, such as a touchscreen display or the like. The display device 234 and/or the input/output device 232 may be used, for example, to allow a user to input information into the wearable medical device 110 and/or receive information from the wearable medical device 110 (e.g., reminders to take a bolus of prescribed medication).

Referring to FIGS. 1-2, the network interface hardware 240 may generally provide the wearable medical device 110 with an ability to interface with one or more external components, such as, for example, the computing device 120, the server 130, and/or the like. Communication with external devices may occur using various communication ports (not shown). An illustrative communication port may be attached to a communications network, such as the Internet, an intranet, a local network, a direct connection, and/or the like. In some embodiments, the network interface hardware 240 may include and/or communicate with any wired or wireless networking hardware, including an antenna, a modem, a LAN port, a wireless fidelity (Wi-Fi) card, a WiMax card, a long term evolution (LTE) card, a ZigBee card, a Bluetooth chip, a USB card, mobile communications hardware, and/or other hardware for communicating with other networks and/or devices.

In some embodiments, a hybrid antenna may be included that combines a radio frequency antenna (e.g., a Bluetooth antenna or GPS antenna, with an inductive loop, such as may be used in a near-field communications (NFC) tag or in an inductive charging system). The functionality for two different systems may be provided in one integrated system. In a hybrid antenna, an inductive loop may be placed in close proximity to the radiator of an inverted-F antenna. The inductive loop may inductively couple with the radiator, allowing the inductive loop to serve as a planar element of the antenna for radio-frequency purposes, thus forming a planar inverted-F antenna. The inductive loop may also serve its normal function (e.g., such as providing current to an NFC chip through inductive coupling with an electromagnetic field generated by an NFC reader).

The autosampler 260 is a device that mechanically obtains a biological sample from a subject. The mechanical operation of an autosampler 260 is typically powered by electricity such as by a battery (not shown). The wearable medical device 110 therefore typically includes a battery, and the autosampler 260 (as well as the other components described herein with respect to FIG. 2) is typically in electrical communication with the battery. For example, the autosampler 260 may include a motor (not shown), and the motor may be in electrical communication with the battery such that electricity from the battery can power the motor to perform an autosampling function of the autosampler 260 and thereby obtain a biological sample from a subject. The autosampler 260 need not necessarily require a motor, for example, because the autosampler 260 may be powered by fluid dynamics such as capillary action and/or the evaporation of water. Illustrative examples of autosamplers include the devices described in U.S. Pat. Nos. 5,356,420, 5,582,184, 5,736,103, 7,976,492, 8,337,421, 9,144,401, each of which is hereby incorporated by reference.

Still referring to FIG. 2, the autosampler 260 may include a needle (not shown) in some embodiments. In embodiments, the needle may include a central lumen that is in fluid communication with an analyte detector 270. Thus, the autosampler 260 may function by drawing a biological sample through the central lumen of the needle such that the biological sample is provided from the needle to the analyte detector 270. Such embodiments may require insertion of the needle into a skin of a subject to draw the biological sensor. In some embodiments, the wearable medical device 110 lacks a catheter.

Still referring to FIG. 2, the analyte detector 270 is typically configured to measure a concentration or relative concentration of an analyte in a biological sample such that the wearable medical device 110, or a computer associated with a wearable medical device 110 (e.g., the computing device 120 depicted in FIG. 1), can determine whether a subject from whom the biological sample originated received a bolus of a pharmaceutical known to affect the concentration of the analyte in the biological sample a period of time before the biological sample was obtained from the subject. The analyte detector 270 may be configured, for example, to measure the concentration or relative concentration of a pharmaceutical (or the concentration or relative concentration of a metabolite of the pharmaceutical) in a biological sample with sufficient accuracy to determine whether a subject from whom the biological sample was obtained received a bolus of the pharmaceutical within about 30 minutes, 1 hour, 2 hours, 3 hours, 4 hours, 5 hours, 6 hours, 7 hours, 8 hours, 9 hours, 10 hours, 11 hours, 12 hours, 18 hours, or 24 hours prior to the obtaining of the biological sample. Illustrative examples of analyte detectors are described in U.S. Pat. Nos. 4,053,381, 4,240,438, 4,533,346, 4,535,786, 5,372,133, 5,462,064, 5,773,270, 6,579,690, 7,467,003, and 7,608,042, each of which is hereby incorporated by reference.

In some embodiments, the analyte detector 270 may include a detection molecule that specifically binds an analyte. The detection molecule and the analyte typically have a dissociation constant, and the dissociation constant is generally less than 10 μM such as less than 1 μM or less than 100 nM. A detection molecule may include a polypeptide or a nucleic acid in some embodiments. For example, a detection molecule may consist of or include an antibody or the antigen-binding portion of an antibody. A detection molecule may similarly consist of or include an aptamer.

In some embodiments, the analyte detector 270 may include a circuit and an ammeter, ohmmeter, or voltmeter. The circuit is in electrical communication with the ammeter, ohmmeter, or voltmeter. In some embodiments, an electrical signal within the circuit includes characteristics such as a current, a resistance, and a voltage. Each of the current, the resistance, and the voltage includes a magnitude that is measurable by the analyte detector 270. For example, contacting the analyte with the detection molecule changes the magnitude of the current, the resistance, or the voltage to produce a change in magnitude. Accordingly, the analyte detector 270 may be particularly configured to measure the change in magnitude caused by the contact of the analyte with the detection molecule. In some embodiments, the change in magnitude correlates with the concentration of an analyte in a biological sample.

In some embodiments, the analyte detector 270 may include a light source, a reporting molecule, a detection chamber, and a light detector. In such embodiments, the analyte detector 270 is configured such that the concentration of the analyte in the biological sample correlates with a concentration of the reporting molecule in the detection chamber as the reporting molecule absorbs, transmits, scatters, fluoresces, or phosphoresces light. Accordingly, the analyte detector 270 can cause the light source to actuate, thereby irradiating the detection chamber with light from the light source. The analyte detector 270 then may detect light from the detection chamber using the light detector for a period of time after obtaining a biological sample. The amount of light detected by the light detector either correlates or inversely correlates with the concentration of the reporting molecule in the detection chamber.

As depicted in the embodiment of FIG. 2, the autosampler 260 and the analyte detector 270 of the wearable medical device 110 are typically in fluid communication with each other, such as, via a conduit 265 or the like. Fluid communication of the autosampler 260 with the analyte detector 270 via the conduit 265 may allow a biological sample obtained from the autosampler 260 to be transferred to the analyte detector 270 for analysis.

Still referring to FIG. 2, the wearable medical device 110 may further include one or more light emitters 280 (e.g., one or more light sources) and/or one or more light detectors 290 (e.g., photodiodes). In some embodiments, one or more light emitters 280 may be configured to emit light that is directed into a detection chamber. In some embodiments, the one or more light emitters 280 may be configured to emit light upon the skin of the subject, and the one or more light detectors 290 may be configured to sample, acquire, and/or detect corresponding light from the skin of the subject (e.g., reflected, transmitted, and/or emitted light from underneath the skin and/or inside the body of the subject). The wearable medical device 110 including the one or more light emitters 280 may include light detectors 290 arranged in an array or pattern (e.g., configured to enhance or optimize the signal-to-noise ratio and/or reduce or minimize power consumption by the light emitters 280 and light detectors 290). The light detectors 290 may sample, acquire, and/or detect physiological data, which may then be processed or analyzed (for example, by resident processing circuitry of a device) to obtain data that is representative of the pulse or heart rate of the subject. Similarly, the one or more light detectors 290 may be configured to obtain data that is representative of the respiration, heart rate variability, oxygen saturation (SpO2), blood volume, blood glucose, skin moisture, and/or skin pigmentation level of the subject.

In some embodiments, the one or more light emitters 280 may emit light having one or more wavelengths that are specific to an analyte or a reporting molecule such as one or more wavelengths that the analyte or reporting molecule absorbs, transmits, or scatters. In some embodiments, the one or more light emitters 280 may emit light having one or more wavelengths that are specific or directed to a type of physiological data to be collected (e.g., pulse or heart rate and/or a physiological parameter to be assessed or determined). For instance, the one or more light emitters 280 may be configured to emit green light (e.g., to generate data corresponding to the pulse or heart rate of a subject). Similarly, the one or more light emitters 280 may be configured to emit red light to generate data corresponding to SpO2.

The color or wavelength of light emitted by the one or more light emitters 280 (e.g., a LED or set of LEDs) may be modified, adjusted, and/or controlled in accordance with a predetermined type of data to be acquired and/or conditions of operation. For example, light emitted by the one or more light emitters 280 may be adjusted and/or controlled to optimize and/or enhance the “quality” of the data obtained and/or sampled by the one or more light detectors 290. For example, the color of light emitted by a LED may be switched from infrared to green when the skin temperature of a subject or an ambient temperature is cool (e.g., in order to enhance a signal corresponding to cardiac activity).

Referring also to FIG. 1, the wearable medical device 110 may be disposed within a housing 112 or the wearable medical device 110 may include a housing 112. The housing 112 typically includes a protrusion 118 or an opening 114 configured to allow the autosampler 260 to obtain a biological sample.

The housing 112 may optionally include a window 116 (e.g., to facilitate optical transmission between the one or more light emitters 280 and the subject and/or the one or more light detectors 290 and the subject). The window 116 may be configured to permit light (e.g., light of a specific wavelength or range of wavelengths) to be emitted by the one or more light emitters 280 of the wearable medical device 110 onto the skin of the subject. The window 116 may also be configured to permit light (e.g., transmitted, reflected, or emitted light) to pass from the skin of the subject through the window 116 (e.g., to be sampled, measured, and/or detected by the one or more light detectors 290, such as one or more photodiodes).

In some embodiments, circuitry related to the one or more light emitters 280 and/or the one or more light detectors 290 may be disposed in the interior of the wearable medical device 110 and/or in the housing 112 thereof. For example, the circuitry related to the one or more light emitters 280 and/or the one or more light detectors 290 may be disposed underneath or behind a plastic and/or glass layer (e.g., painted with infrared ink) or underneath or behind an infrared lens or filter that permits infrared light to pass, but not light in the human visual spectrum. In some embodiments, the light transmissivity of the window 116 may be invisible to a human eye.

In some embodiments, the wearable medical device 110 may include light pipes and/or other light-transmissive structures (not shown) (e.g., to facilitate the transmission of light from the one or more light emitters 280 to the detection chamber or to facilitate the transmission of light from one or more light emitters 280 to the skin and/or body of a subject). Light may be directed from the one or more light emitters 280 to the detection chamber through light pipes and/or other light-transmissive structures. Light from the detection chamber may be directed back to the one or more light detectors 290 in the wearable medical device 110 through the same or similar structures. Light-transmissive structures may employ a material and/or optical design to facilitate low light loss, thereby improving the signal-to-noise-ratio of a light detector. For example, light-transmissive structures may include a lens to facilitate light collection, and portions of a light-transmissive structure may be coated with or adjacent to reflective materials (e.g., to promote internal reflection of light within light-transmissive structures). Light pipes or other light-transmissive structures may include a material that selectively transmits light having one or more specific or predetermined wavelengths with higher efficiency than others, thereby acting as a bandpass filter. Such a bandpass filter may be tuned to improve the signal of a specific analyte. For example, the wearable medical device 110 may include an In-Mold-Labeling (IML) light-transmissive structure (e.g., wherein the light-transmissive structure includes a material with one or more predetermined or desired optical characteristics to create a specific bandpass characteristic to pass infrared light with greater efficiency than light of other wavelengths, such as light that is visible to the human eye). Similarly, the wearable medical device 110 may include a light-transmissive structure having an optically-opaque portion and an optically-transparent portion. In embodiments, the wearable medical device 110 implementing such a light-transmissive structure may include different light transmissivity properties for different wavelengths (e.g., depending on the direction of light travel through the light-transmissive structure). An optically-opaque material may be reflective to a specific wavelength range so as to more efficiently transport light from the detection chamber back to the one or more light detectors 290 (e.g., which may be of a different wavelength relative to the wavelength of the emitted light).

In some embodiments, one or more reflective structures may be placed in the field of view of the one or more light emitters 280 and/or the one or more light detectors 290. For example, hole(s) that channel light from a light source to the detection chamber and/or from the detection chamber to a one or more light detectors 290 (or through one or more light-transmissive structures that perform such channeling travel) may be covered in a reflective material (e.g., to facilitate light transmission). A reflective material may increase the efficiency with which light is transported to the detection chamber from a light source and/or from the detection chamber back into a light detector. A reflectively-coated hole may be filled in with an optical epoxy or other transparent material to prevent material from entering the wearable medical device 110 while allowing light to be transmitted with low transmission loss.

In some embodiments, a light-transmissive structure may include a mask having an opaque material (e.g., that limits the aperture of a light source and/or light detector). A light-transmissive structure may selectively “define” a volume of the detection chamber that light is emitted into and/or detected therefrom. A mask configuration may be employed or implemented to improve a photoplethysmography signal.

In some embodiments, the one or more light emitters 280 and/or the one or more light detectors 290 may be disposed on a Flat Flex Cable or flexible circuit board. A flexible or pliable substrate (e.g., a Flat Flex Cable or flexible circuit board) may be connected to a second substrate (e.g., a circuit board) within a device having other components disposed thereon (e.g., the data processing circuitry). In some embodiments, the wearable medical device 110 may include a second substrate that is relatively inflexible or non-pliable, fixed within the device, having other circuitry and components (passive and/or active) disposed thereon.

In some embodiments, the wearable medical device 110 may include a differential amplifier (not shown) (e.g., to amplify the relative changes in the output of the one or more light detectors 290). A digital average or digital low-pass filtered signal may be subtracted from the output of the one or more light detectors 290. This modified signal may then be amplified before it is digitized.

As depicted in the embodiments of FIGS. 1 and 2, the wearable medical device 110 is generally adapted to be worn or carried on the body of a subject. For example, the wearable medical device 110 may be configured to be worn on a wrist, arm, ankle, or leg of a subject (e.g., as a watch, band, or bracelet). However, it is contemplated that the wearable medical device 110 may be adapted for other wearable forms, such as, for example, around the neck (e.g., like a necklace), around the head (e.g., like a headband), around or on the torso, or the like.

In some embodiments, the wearable medical device 110 may include a protrusion 118 that extends from an interior side of the wearable medical device 110 to contact the skin of the subject when the subject is wearing the wearable medical device 110. When coupled to a subject, the protrusion 118 may engage the skin of the subject with more force than the rest of the wearable medical device 110. The autosampler 260 may form or be incorporated in a portion of the protrusion 118 in some embodiments. As such, when attached to the body of a subject, the autosampler 260 portion of the protrusion 118 of the wearable medical device 110 may engage the skin with more force than the surrounding device housing 112, thereby providing a more secure physical coupling between the skin and the autosampler 260. The protrusion 118 may thereby be configured to cause sustained contact between the wearable medical device 110 and the skin (e.g., to obtain a biological sample comprising sweat or to puncture the skin to obtain a biological sample comprising blood, blood plasma, blood serum, or interstitial fluid). In some embodiments, the protrusion 118 may also include other sensors that benefit from close proximity and/or secure contact to skin, such as, for example, a skin temperature sensor (e.g., a noncontact thermopile that utilizes an optical window or thermistor joined), a pulse oximeter, a blood pressure sensor, an electromyography (EMG) sensor, or a galvanic skin response (GSR) sensor.

In some embodiments, the wearable medical device 110 may be configured to detect the pulse and/or heart rate of a subject. That is, the one or more light detectors 290 of the wearable medical device 110 may be configured to detect, sense, sample, and/or generate data that may be used to determine information representative of the pulse and/or heart rate of a subject. In some embodiments, the one or more light emitters 280 may also be used to illuminate the subject such that the one or more light detectors 290 can determine the pulse and/or the heart rate of the subject. More specifically, the wearable medical device 110 or the one or more light detectors thereof may use the one or more light emitters 280 (e.g., a LED or laser) to emit or output light into the skin or body of a subject. The one or more light detectors 290 (e.g., photodiodes or phototransistors) are configured to sample, measure, and/or detect a transmission, reflection, or absorption of the light from the body of the subject (e.g., photoplethysmography). Accordingly, the wearable medical device 110 and/or the one or more light detectors 290 thereof may be configured to provide data used to detect the heart rate of a subject and/or whether the subject has a pulse. In some embodiments, the wearable medical device 110 may be configured to detect a pulse and/or heart rate only if certain criteria are met. For example, the wearable medical device 110 may be configured to acquire and/or obtain information related to a pulse or heart rate when data from one or more motion sensors (not shown) indicates that the subject is not moving.

The wearable medical device 110 may be configured to select a data acquisition technique, measurement technique, and or analysis program based on a motion signal (e.g., a synchronous detection approach or non-amplitude-modulated approach). For example, the wearable medical device 110 (or the computing device 120 in communication with the wearable medical device 110) may be configured to alter a sampling rate when motion detector circuitry of the wearable medical device 110 detects or determines that motion is below a threshold (e.g., which may suggest that the subject is distressed).

Ambient light and skin color may make it difficult to extract a pulse or heart rate from a photoplethysmographic signal. The effect of ambient light may be reduced by subtracting the detected light signal obtained when the light source is off from the value obtained when the light source is on. The effect of skin color may be reduced by changing the intensity of the light source, the wavelength of the light source, and/or by calculating a ratio or difference between two signals obtained at two different wavelengths. Additionally, skin color may be input or imaged to calibrate a heart rate algorithm, light source brightness, light source wavelength, and/or receiver gain.

In some embodiments, the wearable medical device 110 may be configured to monitor a blood pressure of a subject. A blood pressure may be calculated based on the signal quality of a wearable medical device 110 utilized to detect the pulse of a subject or other feature of a subject's blood. For example, the energy (e.g., squared sum) of a signal in a frequency band of a wearable medical device 110 (e.g., 0.5 Hz to 4 Hz) may correlate with blood pressure. Thus, the strength or quality of a signal used to measure a feature of a subject may correlate with the blood pressure of the subject.

In some embodiments, the wearable medical device 110 may include a strain gauge, pressure sensor, force sensor, or other contact-indicating sensor (not shown). A signal quality metric (e.g., heart rate signal quality) may be calculated based on data from these contact sensors either alone or in combination with data from the various components of the wearable medical device 110 configured to detect a pulse or measure a heart rate. The blood pressure measured by such components may be a diastolic blood pressure, a systolic blood pressure, or a mean arterial pressure (e.g., an approximate diastolic blood pressure, approximate systolic blood pressure, or approximate mean arterial pressure).

In some embodiments, the wearable medical device 110 may monitor heart rate optically through the one or more light detectors 290 when arranged in an array, such as a grid of photodiodes or a CCD camera. For example, the motion of a wearable medical device 110 with respect to the skin of a subject may be tracked through feature-tracking of the skin and/or adaptive motion correction (e.g., using an accelerometer and gyroscope). The one or more light detectors 290 arranged in the array may be in contact with the skin or offset at a small distance away from the skin. The one or more light detectors 290 arranged in the array and its associated optics may be actively controlled (e.g., with a motor) to maintain a stabilized image of the target and acquire a pulse. This optomechanical stabilization may be achieved using information from at least one motion sensor (e.g., a gyroscope) and/or from at least one image feature. In some embodiments, the wearable medical device 110 may be configured to implement relative motion cancellation using a coherent or incoherent light source and emitted by the one or more light emitters 280. The one or more light emitters 280 may be configured to illuminate the skin and the one or more light detectors 290 arranged in the array, with each one of the one or more light detectors 290 associated with comparators for comparing the intensity between neighboring detectors. The wearable medical device 110 may be configured to obtain a “speckle pattern,” which may be tracked using a variety of image tracking techniques such as optical flow, template matching, and/or edge tracking.

In some embodiments, the wearable medical device 110 may be configured to measure an electrocardiograph of a subject. That is, the wearable medical device 110 may include at least three electrodes (not shown). For example, the wearable medical device 110 may include a first electrode that may be configured to contact the skin of the left arm of the subject, a second electrode that may be configured to contact the skin of the right arm of the subject, and a third electrode that may be configured to contact the skin of a leg of the subject. In some embodiments, wearable EKG devices may be used (e.g., U.S. Pat. No. 8,954,129, hereby incorporated by reference in its entirety), including wearable EKG devices that may be configured to connect with another device via a wired connection interface and/or a wireless connection interface (e.g., US 2015/0057512, hereby incorporated by reference in its entirety).

In some embodiments, the wearable medical device 110 may be configured to measure a temperature (e.g., a temperature of a subject). That is, the wearable medical device 110 may be a temperature sensor or the wearable medical device 110 may include a temperature sensor. For example, the wearable medical device 110 may include a skin temperature sensor (e.g., a thermopile or thermistor joined). In another example, the wearable medical device 110 may include a thermocouple or a plurality of thermocouples. In some embodiments, the wearable medical device 110 is configured to measure a temperature of the environment of a subject (e.g., ambient air temperature).

In some embodiments, the monitoring system 100 may be configured to automatically detect or determine whether the wearable medical device 110 is attached to, disposed on, and/or being worn by a subject. For example, the one or more light detectors 290 of the wearable medical device 110 may be used to determine whether the wearable medical device 110 is attached to, disposed on, and/or being worn by a subject. In some embodiments, the computing device 120 associated with the wearable medical device 110 may be configured to interpret data received from the one or more light detectors 290 as indicative that the one or more light detectors 290 are not attached to, disposed on, or otherwise being worn by the subject if the one or more light detectors 290 provides a low return signal. In some embodiments, the wearable medical device 110 may include an infrared detector and/or capacitive detector (not shown). The infrared detector or capacitive detector may be configured to detect or determine whether the subject is in contact with the wearable medical device 110.

It should be understood that the components illustrated in FIG. 2 are merely illustrative and are not intended to limit the scope of this disclosure. More specifically, while the components in FIG. 2 are illustrated as residing within the wearable medical device 110, this is a nonlimiting example. In some embodiments, one or more of the components may reside external to wearable medical device. Similarly, one or more of the components may be embodied in other devices not specifically described herein.

Sensors or electronic systems of the wearable medical device 110 may be integrated with one another or may share components or resources. For example, the one or more light detectors 290 for an optically-based heart rate sensor may also serve as a light detector for determining ambient light level, such as may be used to correct for the effects of ambient light on the heart rate sensor reading. For example, if the one or more light emitters 280 for such a heart rate detector is turned off, the light that is measured by the one or more light detectors 290 may be indicative of the amount of ambient light that is present.

Referring now to FIG. 3, the computing device 120 may include a local interface 300 (e.g., a bus) that communicatively interconnects the various components, including, but not limited to, a processing device 310, memory 320, input/output hardware 330, network interface hardware 340, and/or a data storage device 350.

The local interface 300 is formed from any medium that transmits a signal. As non-limiting examples, the local interface 300 is formed of conductive wires, conductive traces, optical waveguides, or the like. The local interface 300 may also refer to the expanse in which electromagnetic radiation and their corresponding electromagnetic waves are propagated. Moreover, the local interface 300 may be formed from a combination of mediums that transmit signals. In one embodiment, the local interface 300 includes a combination of conductive traces, conductive wires, connectors, and buses that cooperate to permit the transmission of electrical data signals to and from the various components of the computing device 120. Additionally, it is noted that the term “signal” means a waveform (e.g., electrical, optical, magnetic, mechanical or electromagnetic) that travels through a medium, such as DC, AC, sinusoidal-wave, triangular-wave, square-wave, vibration, and the like.

The processing device 310, such as one or more computer processing units (CPU), may be the central processing unit of the computing device 120, performing calculations and logic operations required to execute a program. The processing device 310, alone or in conjunction with one or more of the other elements disclosed in FIG. 3, is an illustrative processing device, computing device, processor, or combination thereof, as such terms are used in this disclosure. As a non-limiting example, processing device 310 may be one of a shared processor circuit, dedicated processor circuit, or group processor circuit. As described herein, the term “shared processor circuit” refers to a single processor circuit that executes some or all machine-readable instructions from the multiple modules. As described herein, the term “group processor circuit” refers to a processor circuit that, in combination with additional processor circuits, executes some or all machine-executable instructions from the multiple modules of one or more non-transitory computer-readable mediums. References to multiple processor circuits encompass multiple processor circuits on discrete dies, multiple processor circuits on a single die, multiple cores of a single processor circuit, multiple threads of a single processor circuit, or a combination of the above.

The memory 320, such as read only memory (ROM) and random access memory (RAM), may constitute illustrative memory devices (i.e., non-transitory, processor-readable storage media). As a non-limiting example, the memory 320 may be one of a shared memory circuit, dedicated memory circuit, or group memory circuit. As described herein, the term “shared memory circuit” refers to a single memory circuit that stores some or all machine-readable instructions from multiple modules, which are described below in further detail. As described herein, the term “group memory circuit” refers to a memory circuit that, in combination with additional memories, stores some or all machine-readable instructions from the multiple modules. Non-limiting examples of the memory 320 include random access memory (including SRAM, DRAM, and/or other types of random access memory), read-only memory (ROM), flash memory, registers, compact discs (CD), digital versatile discs (DVD), and/or other types of storage components.

The memory 320 may include one or more programming instructions thereon that, when executed by the processing device 310, cause the processing device 310 to complete various processes, such as the processes described herein. Optionally, the program instructions may be stored on a tangible computer-readable medium such as a digital disk, flash memory, a memory card, a USB drive, an optical disc storage medium (e.g., Blu-ray™, CD, DVD), and/or other non-transitory processor-readable storage media.

In some embodiments, the program instructions contained on the memory 320 may be embodied as a plurality of software modules, where each module provides programming instructions for completing one or more tasks. For example, as shown in FIG. 3, the memory 320 may contain one or more of operating logic 322, concentration determination logic 324, and communications logic 326. The operating logic 322 may include an operating system and/or other software for managing components of the computing device 120. The concentration determination logic 324 may include programming instructions for determining a concentration of an analyte in a biological sample using various components of the wearable medical device 110 (FIG. 2) and/or determining a meaning of a determined concentration, as described in greater detail herein. The communications logic 326 may include programming instructions for facilitating communications between the computing device 120 and the various other components of the monitoring system 100 (FIG. 1). It should be understood that the various logic modules described herein with respect to FIG. 3 are merely illustrative, and that other logic modules, including logic modules that combine the functionality of two or more of the modules described hereinabove, may be used without departing from the scope of the present application.

Still referring to FIG. 3, the data storage device 350, which may generally be a storage medium that is separate from the memory 320, may contain a data repository for storing electronic data and/or the like relating to a configuration of the computing device 120, information pertaining to a determined concentration and results of analysis, and/or the like. The data storage device 350 may be any physical storage medium, including, but not limited to, a hard disk drive (HDD), memory (e.g., flash memory), removable storage, and/or the like. While the data storage device 350 is depicted as a local device, it should be understood that the data storage device 350 may be a remote storage device that is remotely located from the computing device 120, such as, for example, a server computing device or the like (e.g., the server 130 of FIG. 1). In some embodiments, the data storage device 350 may include or may be coupled to one or more database servers that support NoSQL, MySQL, Oracle, SQL Server, NewSQL, or the like.

Still referring to FIG. 3, illustrative data that may be contained within the data storage device 350 may include, for example, concentration data 352, sensor data 354, configuration data 356, and/or other data 358. Concentration data 352 may include, for example, information pertaining to a determination of a particular concentration of an analyte in a biological sample, as well as data pertaining to a meaning of the particular concentration. The sensor data 354 may include, for example, data relating to information that is sensed by the analyte detector 270 (FIG. 2). Still referring to FIG. 3, the configuration data 356 may include, for example, data relating to configuring the computing device 120.

The input/output hardware 330 may include a basic input/output system (BIOS) that interacts with hardware of the computing device 120 (such as, for example, an input/output device 332 and/or a display device 334), device drivers that interact with particular components of the computing device 120, one or more operating systems, user applications, background services, background applications, etc. The display device 334 is generally any liquid crystal display (LCD), light emitting diode (LED) display, electronic ink (e-ink) display, or the like that can display information to a user. In some embodiments, the display device 334 may be configured as an interactive display that can receive user inputs (e.g., a touch screen display or the like). The input/output device 332 may be hardware components that receive inputs from a user and transmit signals corresponding to the inputs, such as a keyboard, a mouse, a joystick, a touch screen, a remote control, a pointing device, a video input device, an audio input device, a haptic feedback device, and/or the like. In some embodiments, the display device 334 and the input/output device 332 may be combined into a single device, such as a touchscreen display or the like. The display device 334 and/or the input/output device 332 may be used, for example, to allow a user to input information into the computing device 120 and/or receive information from the computing device 120 (e.g., information pertaining to whether a particular subject is taking a bolus of a prescribed medication on a regular basis).

Referring to FIGS. 1 and 3, the network interface hardware 340 may generally provide the computing device 120 with an ability to interface with one or more external components, such as, for example, the wearable medical device 110, the server 130, and/or the like. Communication with external devices may occur using various communication ports (not shown). An illustrative communication port may be attached to a communications network, such as the Internet, an intranet, a local network, a direct connection, and/or the like. In some embodiments, the network interface hardware 340 may include and/or communicate with any wired or wireless networking hardware, including an antenna, a modem, a LAN port, a wireless fidelity (Wi-Fi) card, a WiMax card, a long term evolution (LTE) card, a ZigBee card, a Bluetooth chip, a USB card, mobile communications hardware, and/or other hardware for communicating with other networks and/or devices.

Referring generally to FIGS. 1-3, a method described herein typically includes obtaining a biological sample from a subject. An autosampler of a medical device typically obtains the biological sample. The biological sample may be, for example, blood, blood plasma, blood serum, sweat, or interstitial fluid.

A subject is typically receiving a pharmaceutical that is formulated for self-administration. The subject may actually self-administer the pharmaceutical, or a caregiver may administer the pharmaceutical to the subject. The pharmaceutical may optionally be a prescription pharmaceutical.

A method typically includes determining a concentration of an analyte in a biological sample or a relative concentration of the analyte. The analyte may be, for example, a pharmaceutical or a metabolite of the pharmaceutical. In some embodiments, the analyte is not a molecule that naturally occurs in humans, and the analyte is not a molecule that naturally occurs in human food. In some embodiments, the analyte is a molecule that is not known to occur in nature. For example, the analyte can be a synthetic pharmaceutical that does not occur in nature, a metabolite of a synthetic pharmaceutical that does not occur in nature, an engineered biomolecule that does not occur in nature (such as a therapeutic antibody), or a metabolite of an engineered biomolecule that does not occur in nature.

In some embodiments, an analyte is not the pharmaceutical and the analyte is not a metabolite of the pharmaceutical. Blood glucose concentration, for example, can be indicative of whether a subject received a bolus of the pharmaceutical insulin within a period of time, and glucose is not a metabolite of insulin. Androgen or estrogen concentration can similarly be indicative of whether a subject received a bolus of the pharmaceutical lutropin alfa within a period of time, and neither androgens nor estrogens are metabolites of lutrophin alfa. In some embodiments, the analyte is not insulin. In some embodiments, the analyte is not lutrophin alfa.

In some embodiments, a pharmaceutical is a biomolecule that specifically binds a soluble target, and the analyte is the soluble target. For example, the pharmaceutical may be a therapeutic antibody such as an anti-TNFα antibody (e.g., adalimumab or infliximab), and the analyte is or includes an antigen to which the therapeutic antibody specifically binds, such as TNFα.

The analyte detector 270 of the wearable medical device 110 typically determines the concentration or relative concentration of an analyte in a biological sample. Methods of determining the concentrations of analytes are well known. Sensitive methods to determine concentration include immunoassays, for example, in which an antibody specifically binds an analyte thereby allowing the detection of the analyte and measurement of its concentration. Suitable immunoassays include enzyme-linked immunosorbent assays (ELISA), which can be adapted for use in a wearable medical device. ELISA often relies upon enzyme-assisted signal amplification and colorimetric detection using a dye, fluorophore, or light-emitting molecule. An analyte detector may utilize ELISA or rely upon similar scientific principles as ELISA. Other immunoassays may nevertheless allow the detection of an analyte with sufficient precision to determine whether a subject received a bolus of a pharmaceutical, and such immunoassays may or may not include enzyme-assisted signal amplification and/or colorimetric detection. Simple binding between the antigen-binding portion of an antibody and an analyte may be detected either optically or electrochemically using other well-known methods. Specific binding similarly need not require an antibody, for example, as aptamers can be readily identified that bind a wide variety of different analytes at high affinity.

A concentration of an analyte in a biological sample typically correlates or inversely correlates with a probability that a subject received a bolus of a pharmaceutical within a period of time preceding the obtaining of the biological sample. The concentration correlates with the probability, for example, when the analyte is the pharmaceutical of a metabolite thereof. The concentration inversely correlates with probability, for example, when the pharmaceutical is a therapeutic antibody and the analyte is an antigen to which the therapeutic antibody binds.

A method typically includes determining whether a concentration of an analyte in a biological sample suggests that a subject received a bolus of a pharmaceutical within a period of time preceding the obtaining of the biological sample from the subject. In some embodiments, the various components of the wearable medical device 110 may complete such a determination. In other embodiments, the various components of the computing device 120 may make such a determination. In either embodiment, the wearable medical device 110 and/or the computing device 120 generally determines whether the concentration of the analyte suggests that the subject received the bolus of the pharmaceutical within the period of time.

A method typically includes electronically transmitting a message, which indicates that a concentration of an analyte suggests that the subject either (a) received a bolus of a pharmaceutical within the period of time; or (b) did not receive a bolus of the pharmaceutical within the period of time. In some embodiments, one or more components of the wearable medical device 110 may electronically transmit the message. For example, the wearable medical device 110 may optionally transmit the message, for example, on a graphical user interface (e.g., the display device 234) of the wearable medical device 110. In other embodiments, one or more components of the computing device 120 may transmit the message, for example, on the display device 334 and/or to the wearable medical device 110 to display on the display device 234. The message is optionally an alphanumeric message such as text or prose or a graphic, although the nature of the message is not particularly limiting. The message, for example, may include a sound or audible words.

In some embodiments, a method further includes determining whether the concentration of the analyte suggests that the concentration of the pharmaceutical in the subject is higher than desirable. Such methods may also further include electronically transmitting a message that indicates that the concentration of the analyte suggests that the concentration of the pharmaceutical in the subject is higher than desirable.

In some embodiments, a pharmaceutical has a psychoactive effect. In some embodiments, a pharmaceutical is capable of crossing the blood-brain barrier, and the pharmaceutical is a central nervous system stimulant or a central nervous system depressant. In some embodiments, a pharmaceutical is not insulin, an antibiotic, an aminoglycoside, a polypeptide, a fluoroquinolone, a thioamide, cycloserine, or p-aminosalicylic acid. In some embodiments, a pharmaceutical has one or more side effects; the pharmaceutical has an administration regimen; and the one or more side effects reduce the probability that the subject will comply with the administration regimen. In some embodiments, a pharmaceutical has an administration regimen; a subject has a mental condition; and the mental condition reduces the probability that the subject will comply with the administration regimen.

Methods of monitoring an analyte in a biological sample by measuring one or more properties of an electrical circuit may be employed in some embodiments. For example, the methods described in U.S. Pat. Nos. 4,053,381, 4,240,438, 4,533,346, 4,535,786, 5,372,133, 5,462,064, 5,773,270, 6,579,690, 7,467,003, 7,608,042 may be employed, each of which is hereby incorporated by reference.

The method further includes irradiating the detection chamber with light from one or more light emitters 280; the method further includes detecting light from the detection chamber using the one or more light detectors 290 the period of time after obtaining the biological sample; and the amount of light detected by the one or more light detectors 290 either correlates or inversely correlates with the concentration of the reporting molecule in the detection chamber.

In some embodiments, a biological sample includes blood, blood plasma, blood serum, sweat, or interstitial fluid.

In some embodiments, a method includes detecting a feature of the subject. A method may include detecting a feature directly or indirectly. A method may include receiving a signal from an external device (e.g., wherein the signal includes information about the feature).

The wearable medical device 110 may measure or detect biometric information corresponding to a subject. For example, the wearable medical device 110 may measure or detect one or more vital signs.

The subject may be associated with a RFID tag, such as a wearable or implantable RFID tag or a RFID tag associated a hospital bed, and the wearable medical device may include a RFID sensor. Similarly, the subject may be associated with a RFID sensor, and the wearable medical device 110 may include a RFID sensor (not shown).

In some embodiments, a monitoring system 100 may be configured to determine the location of the subject. The term “location” as used herein may refer to a street address, the geographical coordinates of a position, the elevation or altitude of a position, and/or whether a position is inside or outside. A street address may be an approximate street address (e.g., a range of street addresses or cross-streets near the geographical coordinates of the subject) or an exact street address. A street address may or may not include an apartment, unit, suite, or room number.

In some embodiments, a method includes obtaining the location of the subject or wearable medical device 110, i.e., by obtaining the location of the monitoring system 100 or a component thereof. In some embodiments, the methods described herein include transmitting the location of the subject (e.g., to an emergency responder).

A method may include transmitting an emergency message. An emergency message may include, for example, the location of a subject.

In some embodiments, location determination is performed using one or more components of the monitoring system 100 (e.g., the wearable medical device 110 and the computing device 120 or the server 130. For example, the wearable medical device 110 may acquire a short piece of a position-fixing signal from a GNSS satellite to read a rough timestamp and determine the satellite from which it came. This raw signal and/or other raw signals from other satellites may be sent to a remote device (e.g., a secondary computing device, such as the computing device 120) for location calculation. In certain embodiments, the raw signal may be stored on the wearable medical device 110 before being sent to the remote device. The raw signal may only be sent to the remote device if a condition for sending the raw signal is met. For example, a condition for sending the raw signal may be the detection of an appropriate remote device. The wearable medical device 110 may store the raw signal if no remote device is detected. The raw signal may be sent through a short-range, low-power communication protocol. After the wearable medical device 110 sends the raw signal, the remote device may calculate the location using the raw signal. This location may be sent to the wearable medical device 110 either after calculation or after a condition for sending the location is met. The location may be optionally saved on the server 130.

GPS sensors tend to work better when they are outdoors. As such, the wearable medical device 110 may be able to determine whether it is indoors or outdoors based on the difficulty of acquiring one or more satellites signals, based on an ambient light detector signal, and/or a specific light spectrum detected (e.g., UV light may indicate that the subject is outdoors), based on Wi-Fi or Cell tower multilateration, and/or based on characteristics typical of subjects or of the subject of a monitoring system (e.g., the subject may normally be indoors at night when they are sleeping, and the subject may normally be outdoors during a certain period of time when the subject commutes to work). Additionally, the location may be determined by a pairing of sufficient strength with a secondary device. In certain embodiments, when the wearable medical device 110 detects a pairing of a predetermined strength with a secondary device, it may automatically download the location from the secondary device.

In some embodiments, a wearable medical device 110 may be configured to transmit an emergency message to an emergency responder. An emergency responder may be an operator, dispatcher, healthcare provider, nurse, nurses aid, nursing assistant, auxiliary nurse, subject care technician, nurse practitioner, physician, physician assistant, pharmacist, emergency medical technician, firefighter, police officer, security officer, security guard, concierge, personal care provider, home care provider, home health care provider, home health aide/assistant, geriatric aide/assistant, custodial care provider, or social worker, or a secretary, assistant, or answering service provider of any one of the foregoing.

In some embodiments, an emergency responder may be an operator at an emergency dispatch center (e.g., a 911 dispatch center). An emergency dispatch center may be a public or private dispatch center. An emergency responder may be a concierge or security officer. An emergency responder may be a personal assistant. In some embodiments, an emergency responder is an emergency contact, such as a friend, family member, or physician of the subject (e.g., who the subject designated as an emergency contact).

It should now be understood that that the devices, systems, and methods described herein utilize a wearable medical device to monitor subject compliance with a pharmaceutical treatment regimen by obtaining a biological sample from the subject and determining compliance from the sample. In some embodiments, the monitoring system described herein may further include a computing device that splits the duty of carrying out one or more of the processes of determining compliance based on information received from the biological sample with the wearable medical device to minimize the cost of components within the wearable medical device and/or minimize the amount of processing time necessary to make such a determination. In other embodiments, the wearable medical device may complete all of the one or more processes of determining compliance to minimize the amount of data that is transmitted over a network and/or to avoid potential security issues. The wearable medical device generally includes a processing device, memory, an autosampler, and analyte detector that carry out the various steps described herein for monitoring compliance.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A method to monitor compliance with a pharmaceutical treatment regimen, the method comprising:
obtaining, via an autosampler of a wearable medical device, a biological sample from a subject, wherein:
the wearable medical device comprises the autosampler in fluid communication with the analyte detector, and
the subject is receiving a pharmaceutical that is formulated for self-administration;
determining, via the analyte detector of the wearable medical device, a concentration of an analyte in the biological sample, wherein the concentration of the analyte correlates with a probability that the subject received a bolus of the pharmaceutical within a period of time prior to obtaining the biological sample;
determining whether the concentration of the analyte suggests that the subject received the bolus of the pharmaceutical within the period of time; and
electronically transmitting a message indicating that the concentration of the analyte suggests that the subject either:
received the bolus of the pharmaceutical within the period of time, or
did not receive the bolus of the pharmaceutical within the period of time.

2. The method of claim 1, further comprising:
determining whether the concentration of the analyte suggests that the concentration of the pharmaceutical in the subject is higher than a predetermined concentration; and
electronically transmitting a message that indicates that the concentration of the analyte suggests that the concentration of the pharmaceutical in the subject is higher than the predetermined concentration.

3. The method of claim 1, further comprising:
contacting the analyte with a detection molecule in the analyte detector, the detection molecule specifically binding the analyte.

4. The method of claim 3, further comprising:
measuring a change in magnitude of a current, a resistance, or a voltage of a circuit in the analyte detector, wherein the change in magnitude correlates with the concentration of the analyte in the biological sample.

5. The method of claim 1, further comprising detecting an amount of light via a light detector in the analyte detector for a second period of time after obtaining the biological sample, wherein the amount of light either correlates or inversely correlates with the concentration of the reporting molecule.

6. A wearable medical device to monitor compliance with a pharmaceutical treatment regimen, the wearable medical device comprising:
an autosampler;
an analyte detector fluidly coupled to the autosampler;
a processing device communicatively coupled to the analyte detector and the autosampler; and
a non-transitory storage medium communicatively coupled to the processing device, the non-transitory storage medium comprising one or more programming instructions thereon that, when executed, cause the processing device to:
direct the autosampler to obtain a biological sample from a subject that is receiving a pharmaceutical that is formulated for self-administration,
direct the analyte detector to determine a concentration of an analyte in the biological sample, wherein the concentration of the analyte correlates with a probability that the subject received a bolus of the pharmaceutical within a period of time prior to obtaining the biological sample,
determine whether the concentration of the analyte suggests that the subject received the bolus of the pharmaceutical within the period of time, and
transmit a message indicating that the concentration of the analyte suggests that the subject either:
received the bolus of the pharmaceutical within the period of time, or
did not receive the bolus of the pharmaceutical within the period of time.

7. The wearable medical device of claim 6, wherein the one or more programming instructions, when executed, further cause the processing device to:
determine whether the concentration of the analyte suggests that the concentration of the pharmaceutical in the subject is higher than a predetermined concentration; and
electronically transmit a message that indicates that the concentration of the analyte suggests that the concentration of the pharmaceutical in the subject is higher than the predetermined concentration.

8. The wearable medical device of claim 6, wherein:
the analyte detector comprises a detection molecule that specifically binds to the analyte when the analyte is contacted with the detection molecule.

9. The wearable medical device of claim 8, wherein the detection molecule comprises a polypeptide or a nucleic acid.

10. The wearable medical device of claim 9, wherein the detection molecule comprises an antibody, an antigen-binding portion of an antibody, or an aptamer.

11. The wearable medical device of claim 8, wherein:
the analyte detector comprises a circuit;
the analyte detector comprises an ammeter, ohmmeter, or voltmeter;
the circuit is in electrical communication with the ammeter, ohmmeter, or voltmeter;
the circuit comprises a current, a resistance, and a voltage;
each of the current, the resistance, and the voltage comprises a magnitude;
contacting the analyte with the detection molecule changes the magnitude of the current, the resistance, or the voltage to produce a change in magnitude; and
the one or more programming instructions, when executed, further cause the processing device to direct the analyte detector to measure the change in magnitude, the change in magnitude correlating with the concentration of the analyte in the biological sample.

12. The wearable medical device of claim 6, wherein:
the analyte detector comprises a light source, a reporting molecule, a detection chamber, and a light detector;
the analyte detector is configured such that the concentration of the analyte in the biological sample correlates with a concentration of the reporting molecule in the detection chamber;
the reporting molecule absorbs, transmits, scatters, fluoresces, or phosphoresces light; and
the one or more programming instructions, when executed, further cause the processing device to:
direct the analyte detector to cause the light source to irradiate the detection chamber with light from the light source;
direct the analyte detector to cause the light detector to detect light from the detection chamber the period of time after obtaining the biological sample; and
determine that the amount of light detected by the light detector either correlates or inversely correlates with the concentration of the reporting molecule in the detection chamber.

13. The wearable medical device of any one of claim 6, wherein:
the autosampler comprises a needle;
the needle comprises a central lumen;
the central lumen of the needle is in fluid communication with the analyte detector; and
the one or more programming instructions, when executed, further cause the processing device to direct the autosampler to draw the biological sample through the central lumen of the needle.

14. A monitoring system to monitor compliance with a pharmaceutical treatment regimen, the monitoring system comprising:
a wearable medical device comprising an autosampler and an analyte detector fluidly coupled to the autosampler, wherein:
the autosampler is configured to obtain a biological sample from a subject that is receiving a pharmaceutical that is formulated for self-administration, and
the analyte detector is configured to determine a concentration of an analyte in the biological sample, wherein the concentration of the analyte correlates with a probability that the subject received a bolus of the pharmaceutical within a period of time prior to obtaining the biological sample; and
a computing device that is remote from the wearable medical device and communicatively coupled to the medical device, the computing device configured to receive data from the analyte detector corresponding to the determined concentration of the analyte in the biological sample, determine whether the concentration of the analyte suggests that the subject received the bolus of the pharmaceutical within the period of time, and transmit a message indicating that the concentration of the analyte suggests that the subject either:
received the bolus of the pharmaceutical within the period of time, or
did not receive the bolus of the pharmaceutical within the period of time.

15. The monitoring system of claim 14, wherein the analyte detector comprises a detection molecule that specifically binds to the analyte when the analyte is contacted with the detection molecule.

16. The monitoring system of claim 15, wherein the detection molecule comprises a polypeptide or a nucleic acid.

17. The monitoring system of claim 16, wherein the detection molecule comprises an antibody, an antigen-binding portion of an antibody, or an aptamer.

18. The monitoring system of claim 14, wherein:
the analyte detector comprises a circuit;
the analyte detector comprises an ammeter, ohmmeter, or voltmeter;
the circuit is in electrical communication with the ammeter, ohmmeter, or voltmeter;
the circuit comprises a current, a resistance, and a voltage;
each of the current, the resistance, and the voltage comprises a magnitude;
contacting the analyte with the detection molecule changes the magnitude of the current, the resistance, or the voltage to produce a change in magnitude; and
the analyte detector is further configured to measure the change in magnitude, the change in magnitude correlating with the concentration of the analyte in the biological sample.

19. The monitoring system of claim 14, wherein:
the analyte detector comprises a light source, a reporting molecule, a detection chamber, and a light detector;
the concentration of the analyte in the biological sample correlates with a concentration of the reporting molecule in the detection chamber;
the reporting molecule absorbs, transmits, scatters, fluoresces, or phosphoresces light;
the analyte detector is further configured to cause the light source to irradiate the detection chamber with light from the light source, cause the light detector to detect light from the detection chamber the period of time after obtaining the biological sample and determine that the amount of light detected by the light detector either correlates or inversely correlates with the concentration of the reporting molecule in the detection chamber.

20. The monitoring system of claim 14, wherein:
the autosampler comprises a needle;
the needle comprises a central lumen;
the central lumen of the needle is in fluid communication with the analyte detector; and
the autosampler is further configured to draw the biological sample through the central lumen of the needle.
